(19) United States
(12) Patent Application Publication
(10) Pub. No.: US 2026/0232445 A1
(43) Pub. Date: Aug. 13, 2026

(54) SYSTEMS AND METHODS FOR THE TREATMENT OF CHRONIC VENOUS DISEASE

(71) Applicant: CLEARSTREAM TECHNOLOGIES LIMITED, Enniscorthy, County Wexford (IE)

(72) Inventor: Allan RONAN, Enniscorthy, County Wexford (IE)

(21) Appl. No.: 19/155,719

(22) PCT Filed: Feb. 13, 2023
(86) PCT No.: PCT/EP2023/053457

§ 371 Date: Aug. 12, 2025

(51) Int. Cl. A61F 2/24; A61M 1/00; A61M 25/00

(52) U.S. Cl. CPC A61F 2/2475; A61F 2/246; A61F 2/2466; A61M 1/84; A61F 2220/0008; A61F 2230/0065; A61F 2250/001; A61M 2025/0004

(57) ABSTRACT

The present disclosure relates to a system for closing a valve of a vessel or a vessel in a patient's vascular system, said system comprising: a delivery tool for delivering a closing device, and comprising a delivery catheter with a lumen configured to receive at least a portion of the valve therein; and a closing device configured to be coupled to said at least a portion of the valve so as to close the valve.

### TECHNICAL FIELD

The present disclosure relates to systems and methods for the treatment of chronic venous disease and chronic venous insufficiency. The present disclosure also relates to systems and methods for closing valves and vessels of the vascular system.

### BACKGROUND

Varicose veins are dilated, tortuous veins which are associated with structural vessel wall changes, incompetent venous valves, reflux, and pooling of blood. They form part of the spectrum of chronic venous disease (CVD).

The cause of varicose veins is unknown but genetic factors leading to weakness in vein wall components and valves are important in their development. In healthy veins, blood flows towards the heart and is prevented from flowing backwards by a series of valves. The vein walls may lose their elasticity and become stretched, causing the valves to weaken. If the valves are weakened or damaged, blood can leak, flow backwards and collect in the vein, eventually causing it to become swollen and enlarged (varicose).

Patients experience symptoms ranging from heaviness, aching pain, and swelling to skin irritation, discoloration, and ulceration in severe cases. In some cases, blood clots may form in varicose veins and result in complications, such as deep vein thrombosis or thrombophlebitis.

The interventional treatment used to manage varicose veins depends, in part, on the type of vein to be treated. Deep veins are generally essential and cannot be removed. These are therefore treated, for example by narrowing the inner diameter of the vein or by repairing venous valves. By contrast, superficial and perforator veins may be removed or sealed, for example by the surgical removal of the varicose veins, endothermal ablation, and sclerotherapy. Endothermal ablation involves the sealing of veins using heat. Sclerotherapy involves the injection of a chemical solution to shrink and seal veins. In these treatment methods, sealing is achieved by treating and modifying the structure or configuration of the vein sealing site, so that structural integrity and overall suitability of the tissues surrounding the intended sealing site may impact the quality of the seal.

It is an object of this disclosure to provide systems and methods useful to mitigate problems such as those described above and to provide an improved alternative to existing treatments. In particular, there is a need for improved systems and methods to treat chronic venous disease and chronic venous insufficiency. There is also a need for improved systems and methods to safely and simply close or seal valves and/or vessels in the vascular system of a patient. In particular, there is a need for improved systems and methods to close or seal incompetent vessels, such as varicose or otherwise defective veins.

### SUMMARY

According to a first aspect of the disclosure, there is provided a system for closing a valve of a vessel in a patient's vascular system, said system comprising: a delivery tool for delivering a closing device, and comprising a delivery catheter with a lumen configured to receive at least a portion of the valve therein; and a closing device configured to be coupled to said at least a portion of the valve so as to close the valve.

In some embodiments, the vessel is a vein, such as a superficial or perforator vein.

In some embodiments, the valve is a venous valve.

In some embodiments, the portion of the valve is the ends of both valve leaflets.

In some embodiments, the closing device is configured to be releasably mounted to the delivery catheter. In some embodiments, the closing device is configured to be releasably coupled to the outer circumference of the catheter.

In some embodiments, the closing device is releasably mounted to the distal end of the delivery catheter.

In some embodiments, the closing device is moveable from a proximal end to a distal end of the delivery catheter.

In some embodiments, the circumference of the closing device is adjustable.

In some embodiments, the closing device comprises an elastic material, an elastomeric material, and/or a shape-memory material.

In some embodiments, the closing device is a closing ring. The closing device consists or comprises a sealing ring, such as an open-ring or a closed-ring.

In some embodiments, the closing device is integrally formed.

In some embodiments, the closing device comprises an adhesive coating.

In some embodiments, the delivery tool comprises a release device for releasing the closing device from the delivery tool, said release device being configured to slide axially along the delivery catheter.

In some embodiments, the release device is a release catheter.

In some embodiments, the delivery tool comprises a placement device configured to push the closing device from a proximal end of the delivery catheter to the distal end of the delivery catheter, said placement device being configured to slide axially along the delivery catheter.

In some embodiments, the placement device is a placement catheter.

In some embodiments, the delivery tool comprises an outer catheter concentrically positioned around the delivery catheter.

In some embodiments, the system further comprises a suction tool.

In some embodiments, the suction tool is a suction line.

In some embodiments, the suction tool comprises a first suction line arranged and configured to draw at least a portion of the valve into the lumen of the delivery catheter.

In some embodiments, the suction tool comprises a second suction line for drawing contents from the vessel.

In some embodiments, the suction tool comprises a pump.

In some embodiments, the system further comprises a delivery sheath configured to receive the delivery tool therein.

In some embodiments, the system comprises a control device for controlling the release device and/or the placement device.

In some embodiments, the control device is positioned at the proximal end of the system.

In some embodiments, the control device is housed in a handle.

According to another aspect of the present disclosure, there is provided a system for closing a valve of a vessel in a patient's vascular system, said system comprising a delivery catheter with a lumen configured to receive at least a portion of the valve therein; and a closing device configured to be coupled to said at least a portion of the valve so as to close the valve

According to another aspect of the present disclosure, there is provided a method for closing a valve of a patient's vascular system, said method comprising the steps of: delivering a closing device using a delivery tool comprising a delivery catheter with a lumen configured to receive at least a portion of the valve therein; and closing the valve using a closing device configured to be coupled to said at least a portion of the valve.

In some embodiments, the vessel is a vein, such as a superficial or perforator vein. In some embodiments, the valve is a venous valve. In some embodiments, the portion of the valve is the ends of both valve leaflets.

In some embodiments, the method further comprises the step of releasably mounting the closing device to the delivery catheter. In some embodiments, the closing device is releasably coupled to the outer circumference of the catheter. In some embodiments, the closing device is releasably mounted to the distal end of the delivery catheter.

In some embodiments, the method further comprises the step of moving the closing device from a proximal end to a distal end of the delivery catheter. In some embodiments, the step of moving the closing device is performed using a placement device as described hereinabove in relation to the first aspect.

In some embodiments, the method further comprises the step of releasing the closing device from the delivery tool. In some embodiments, the step of releasing the closing device is performed using a release device as described hereinabove in relation to the first aspect.

In some embodiments, the delivery tool comprises an outer catheter concentrically positioned around the delivery catheter.

In some embodiments, the method further comprises the step of drawing at least a portion of the valve into the lumen of the delivery catheter. In some embodiments, the step of drawing at least a portion of the valve into the lumen of the delivery catheter is performed using a suction tool as described hereinabove in relation to the first aspect.

In some embodiments, the method further comprises the step of drawing contents from the vessel. In some embodiments, the step of drawing contents from the vessel is performed using a suction tool as described hereinabove in relation to the first aspect.

In some embodiments, the step of delivering the closing device comprises placing the delivery tool in a delivery catheter.

In some embodiments, the method further comprises the step of controlling the release device and/or the placement device. In some embodiments, the step of controlling the release device and/or the placement device is performed using a control device as described hereinabove in relation to the first aspect.

According to another aspect of the present disclosure, there is provided a system for closing a vessel of a patient's vascular system, said system comprising: a delivery tool for delivering a closing device, and comprising a delivery catheter with a lumen configured to receive at least a portion of the vessel therein; and a closing device configured to be coupled to said at least a portion of the vessel so as to close the vessel.

In some embodiments, the vessel is a vein, such as a superficial or perforator vein.

In some embodiments, the portion of the vessel is a portion of the circumferential vessel wall. In some embodiments, the portion of the vessel is a circumference of the vessel wall.

In some embodiments, the closing device is configured to be releasably mounted to the delivery catheter. In some embodiments, the closing device is configured to be releasably coupled to the outer circumference of the catheter.

In some embodiments, the closing device is releasably mounted to the distal end of the delivery catheter.

In some embodiments, the closing device is moveable from a proximal end to a distal end of the delivery catheter.

In some embodiments, the circumference of the closing device is adjustable.

In some embodiments, the closing device comprises an elastic material, an elastomeric material, and/or a shape-memory material.

In some embodiments, the closing device consists of or comprises a sealing ring, such as an open-ring or a closed-ring.

In some embodiments, the closing device is integrally formed.

In some embodiments, the closing device comprises an adhesive coating.

In some embodiments, the delivery tool comprises a release device for releasing the closing device from the delivery tool, said release device being configured to slide axially along the delivery catheter.

In some embodiments, the delivery tool comprises a placement device configured to push the closing device from a proximal end of the delivery catheter to the distal end of the delivery catheter, said placement device being configured to slide axially along the delivery catheter.

In some embodiments, the delivery tool comprises an outer catheter concentrically positioned around the delivery catheter.

In some embodiments, the system further comprises a suction tool.

In some embodiments, the suction tool comprises a first suction line arranged and configured to draw at least a portion of the vessel into the lumen of the delivery catheter.

In some embodiments, the suction tool comprises a second suction line for drawing contents from the vessel.

In some embodiments, the system further comprises a delivery sheath configured to receive the delivery tool therein.

In some embodiments, the system comprises a control device for controlling the release device and/or the placement device.

In some embodiments, the control device is positioned at the proximal end of the system.

In some embodiments, the control device is housed in a handle.

According to another aspect of the present disclosure, there is provided a method for closing a vessel of a patient's vascular system, said method comprising the steps of: delivering a closing device using a delivery tool comprising a delivery catheter with a lumen configured to receive at least a portion of the vessel therein; and closing the vessel using a closing device configured to be coupled to said at least a portion of the vessel.

In some embodiments, the vessel is a vein, such as a superficial or perforator vein.

In some embodiments, the method further comprises the step of releasably mounting the closing device to the delivery catheter. In some embodiments, the closing device is releasably coupled to the outer circumference of the catheter. In some embodiments, the closing device is releasably mounted to the distal end of the delivery catheter.

In some embodiments, the method further comprises the step of moving the closing device from a proximal end to a distal end of the delivery catheter. In some embodiments, the step of moving the closing device is performed using a placement device as described hereinabove in relation to the third aspect.

In some embodiments, the method further comprises the step of releasing the closing device from the delivery tool. In some embodiments, the step of releasing the closing device is performed using a release device as described hereinabove in relation to the third aspect.

In some embodiments, the delivery tool comprises an outer catheter concentrically positioned around the delivery catheter.

In some embodiments, the method further comprises the step of drawing at least a portion of the vessel into the lumen of the delivery catheter. In some embodiments, the step of drawing at least a portion of the vessel into the lumen of the delivery catheter is performed using a suction tool as described hereinabove in relation to the third aspect.

In some embodiments, the portion of the vessel is a portion of the circumferential vessel wall. In some embodiments, the portion of the vessel is a circumference of the vessel wall.

In some embodiments, the method further comprises the step of drawing contents from the vessel. In some embodiments, the step of drawing contents from the vessel is performed using a suction tool as described hereinabove in relation to the third aspect.

In some embodiments, the step of delivering the closing device comprises placing the delivery tool in a delivery catheter.

In some embodiments, the method further comprises the step of controlling the release device and/or the placement device. In some embodiments, the step of controlling the release device and/or the placement device is performed using a control device as described hereinabove in relation to the third aspect.

According to another aspect of the present disclosure, there is provided a method for treating chronic venous disease, said method comprising the steps of: delivering a closing device using a delivery tool comprising a delivery catheter with a lumen configured to receive at least a portion of the valve therein; and closing the valve using a closing device configured to be coupled to said at least a portion of the valve.

According to another aspect of the present disclosure, there is provided a method for treating chronic venous disease, said method comprising the steps of: delivering a closing device using a delivery tool comprising a delivery catheter with a lumen configured to receive at least a portion of the vessel therein; and closing the vessel using a closing device configured to be coupled to said at least a portion of the vessel.

According to another aspect of the present disclosure, there is provided a method for manufacturing a system as described hereinabove, comprising the step of coupling the closing device to the delivery catheter.

These and other features of the concepts provided herein will become more apparent to those of skill in the art in view of the accompanying drawings and the following description, which describe particular embodiments of such concepts in greater detail.

### BRIEF DESCRIPTION OF THE DRAWINGS

FIG. 1 is a schematic cross-sectional representation of a target valve in a vessel of a patient's vascular system;

FIG. 2 is a schematic sectional representation of a system according to the present disclosure, positioned adjacent the leaflets of the valve of FIG. 1;

FIG. 3 is a schematic sectional representation of the system of FIG. 2, with an activated suction tool;

FIG. 4 is a schematic sectional representation of the system of FIG. 2, with an activated release device; and

FIG. 5 is a schematic cross-sectional representation of the target valve closed with a closing device.

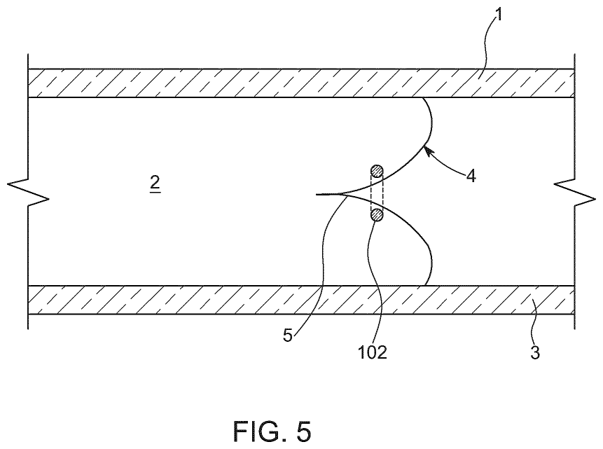

### DETAILED DESCRIPTION

The embodiments described herein are provided as exemplary and non-limiting embodiments of the present invention.

Within the context of the present disclosure, “proximal”, “a proximal end”, “a proximal portion”, “a proximal end portion”, and “a proximal length”, of for example a delivery tool, designate an end, portion, end portion, length of the tool intended to be near a clinician when the delivery tool is used on a patient. Within the context of the present disclosure, “distal”, “a distal end”, “a distal portion”, “a distal end portion”, and “a distal length”, of for example a delivery tool, designate an end, portion, end portion, length of the tool intended to be near or in a patient when the catheter is used on a patient. In the accompanying figures, the proximal side is designated by P and the distal side is designated by D.

Withing the context of the present disclosure, “a proximal end” and a “distal end”, of for example a catheter, designate the proximal terminal portion and the distal terminal distal portion of the catheter.

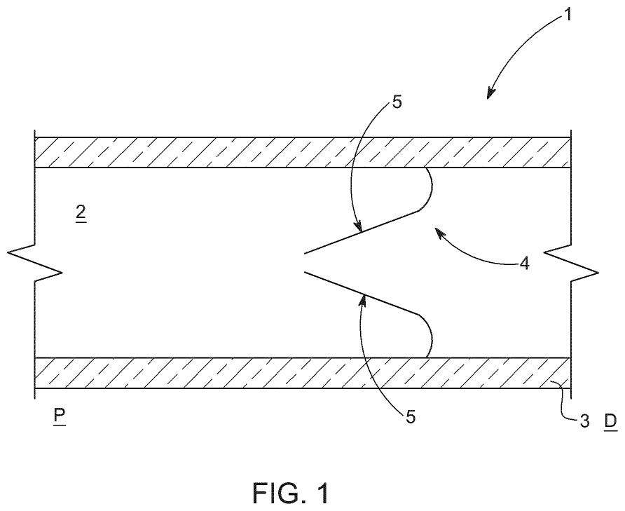

FIG. 1 illustrates a vessel 1 of a patient's vascular system. The vessel 1 may be a superficial vein or a perforator vein. The vessel 1 has a lumen 2 and a wall 3 surrounding the lumen 2. The vessel 1 comprises a valve 4 with valve leaflets 5.

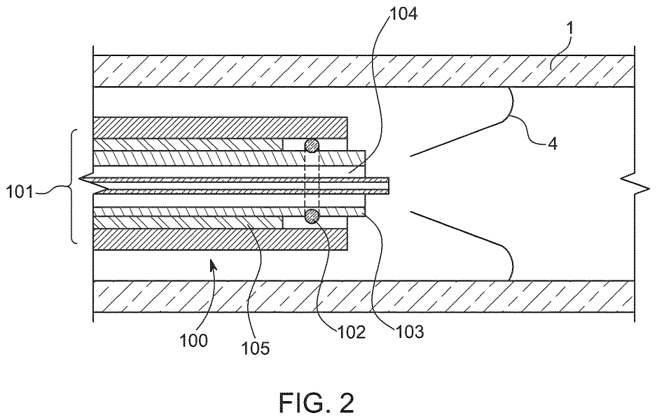

With reference to FIG. 2, there is illustrated a system 100 for closing a valve 4 of a vessel 1 in a patient's vascular system. The system 100 comprises a delivery tool 101 for delivering a closing device 102. The delivery tool 101 comprises a delivery catheter 103 with a lumen 104 configured to receive at least one portion of the valve 4 in the lumen 104 of the delivery catheter 103; and a closing device 102 configured to be coupled to said at least one portion of the valve 4 so as to close the valve 4. In this embodiment, the at least one portion of the valve 4 may be the leaflets 5 of the valve 4. In other words, the closing device 2 is configured to be coupled to the valve leaflets 5 so as to close the valve 4.

The system 100 will be described in further details hereinbelow with reference to the closure of a valve 4 of a vessel 1 in a patient's vascular system.

### The Closing Device

The closing device 102 is configured to be coupled to at least one portion of the valve 4 so as to close or seal the valve 4. That is, the closing device 102 is configured to be coupled to that portion of the valve 4 which is received in the delivery catheter 103. In its working configuration, the closing device 102 is located within the vessel 1, so that the system 100 allows the valve 4 to be closed from within the vessel 1. This is a departure from conventional systems configured close the vessel by positioning a sealing device or clip around the outer circumference of the vessel. Such clips may be implanted surgically, or using a catheter system. In the latter case, the clip is delivered through the vascular system, and an opening must be created through the vessel wall in order to position the clip around the vessel. The system 100 according to the present disclosure is minimally invasive compared to surgical procedures, and less complex in that closure is effected from within the patient's vasculature.

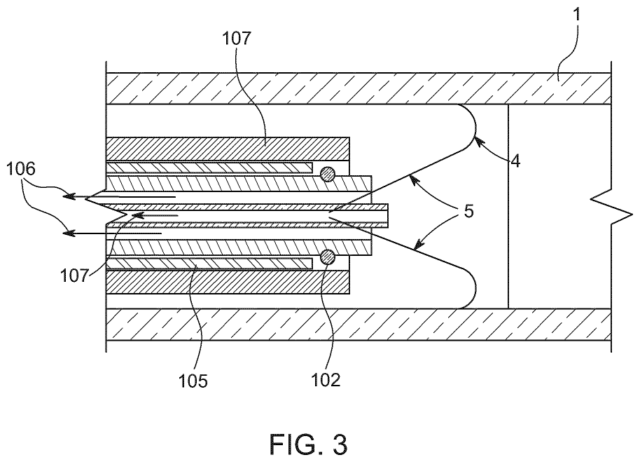

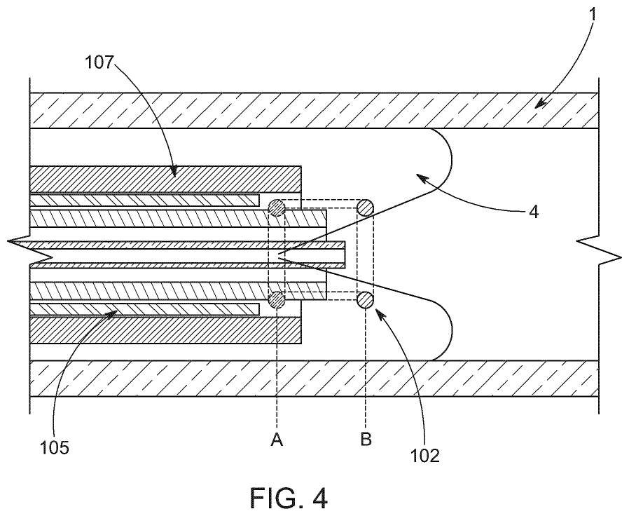

In the embodiment illustrated in FIGS. 3 and 4, the at least one portion of the valve 4 is or comprises the leaflets 5 of the valve 4. That is, the closing device 102 is configured to be coupled to the valve leaflets 5 so as to close the valve 4. Coupling may be effected by bringing the leaflets 5 closer to each other, into closing or sealing contact. The present system 100 effectively seals the valve 4, and hence the vessel 1, without the need for supplementary closing means and without the need for suture.

The closing device 102 may be configured to mechanically close the valve 4. The closing device 102 may consist of or comprise a sealing ring, such as an open ring or a closed-ring, a band, a clip, or any other mechanical means suitable to close the valve 4 and vessel 1. The closing device 102 may be integrally formed.

The circumference or an inner dimension of the closing device 102 may be adjustable. For example, the circumference may have a first configuration in which the closing device 102 partially or completely surrounds but does not contact the at least one portion (e.g., the leaflets 5) of the valve 4, and a second configuration in which the closing device 102 partially or completely surrounds the leaflets 5 of the valve 4 in closing contact. The closing device 102 may comprise a flexible material, an elastic material, an elastomeric material, and/or a shape-memory material.

The closing device 102 may comprise an adhesive coating, for example an external layer of adhesive material, so as to secure the closing device 102 to the at least one portion of the valve 4. This adhesive coating minimises or prevents displacement of the closing device 102.

The closing device 102 may be configured to be releasably mounted to the delivery catheter 103 of the delivery tool 101 of the system 100. For example, the closing device 102 is configured to partially or completely surround the outer circumference of the delivery catheter 103. The closing device 102 may be releasable retained by the delivery catheter 103 by frictional forces resulting from the composition, configuration and/or relative dimensions of the closing device 102 relative to the delivery catheter 103.

The closing device 102 may be releasably mounted to the distal end of the delivery catheter 103. Additionally or alternatively, the closing device 102 may be moveable from a proximal end to a distal end of the delivery catheter 103. Therefore, the closing device 102 may be mounted to the distal end of the delivery catheter 103 prior to inserting the delivery tool 101 in the patient, or the delivery tool 101 may be inserted in the patient and the closing device 102 subsequently coupled to the proximal end of the delivery catheter 103 and pushed to the distal end of the delivery catheter 103.

### The Delivery Tool

The delivery tool 101 may comprise a placement device configured to push the closing device 102 from a proximal end of the delivery catheter 103 to the distal end of the delivery catheter 103. Additionally or alternatively, delivery tool 101 may comprise a release device configured to release the closing device 102 from the delivery catheter 103, for example from the distal end of the delivery catheter 103

As illustrated in FIG. 2, the delivery tool 101 may comprise a delivery catheter 103, and a placement and/or release device embodied by a single release catheter 105. Within the context of the present disclosure, it is envisaged to provide a placement catheter and a separate release catheter for the placement and release, respectively, of the closing device 102. In the present embodiment, the release catheter 105 is configured to place and release the closing device 102.

The release catheter 105 may be configured to slide axially along the delivery catheter. For example, the release catheter 105 is an outer catheter concentrically positioned around the delivery catheter 103, and which can slide relative to the delivery catheter 103.

As illustrated in FIG. 3, the system 100 may comprise a suction tool. This suction tool may comprise a first suction line 106 arranged and configured to draw at least a portion of the valve 4 into the delivery tool 101, for example into the lumen 104 of the delivery catheter 103. The first suction line 106 may be connected to an external vacuum pump (not shown) and the vacuum force may be controlled and adjusted by an external vacuum control device. In this embodiment, the first suction line 106 is in the lumen 104 of the delivery catheter 103 and draws the leaflets 5 of the valve 4 into the lumen 104 of the delivery catheter 103. This allows the leaflets 5 to be positioned and releasably secured into the lumen 104 of the delivery catheter 103 so as to be fitted with a closing device 102.

The suction tool of the system 100 may comprise a second suction line 107 for drawing contents from the vessel 1. The content of the vessel 1 may comprise fluids (blood) and solids (debris, tissues). Whilst some contents may be removed via the first suction line 106, the second suction line 107 allows the further removal of contents which may obstruct visual imaging and prevent accurate delivery, positioning, and securing of the closing device 102. The second suction line 107 may be connected to an external vacuum pump (not shown) and the vacuum force may be controlled and adjusted by an external vacuum control device. The external vacuum pump and external vacuum control device may be the same as or different from that connected to the first suction line 106.

The second suction line 107 may be located in the delivery catheter 103, for example in the lumen 104 of the delivery catheter 103, axially positioned in or adjacent or against the inner wall of the delivery catheter 103. Alternatively, the second suction line 107 may be located outside the delivery catheter 103, between the release catheter 105 and the delivery catheter 105 or outside the release catheter 105.

The system 100 may further comprise a delivery sheath 108 configured to receive the (components of the) delivery tool 101. The delivery tool 101 may be slidably received within the lumen of delivery sheath 108.

The system 100 may comprise a control device (not shown), which may be external control device. The control device may control any one or more of the delivery catheter 103, the placement device, the release device, the suction tool, the first suction line 106, the second suction line 107. The control device may be housed in a handle, for example a handle coupled to the delivery tool 101.

In order to assist the clinician in the placement of the closing device 102, the system 100 may comprise one or more imaging markers. These markers may be positioned at the distal end of any one or more of the delivery catheter 103, the placement device, the release device, the suction tool, the first suction line 106, the second suction line 107. Additionally or alternatively, one or more of the delivery catheter 103, the placement device, the release device, the suction tool, the first suction line 106, the second suction line 107 may comprise a radio-opaque material.

The system 100 may comprise means to treat the closing site. For example, the system may comprise means for delivering one or more drugs to the closing site, such as drugs to promote tissue healing and growth.

The system 100 may comprise one or more occlusion devices for occluding the distal side of the target closing site (e.g., valve 4), so that the target closing site is located between the distal end of the delivery tool 101 and an occlusion device. Occlusion devices are known in the art and will not be described herein for the sake of conciseness.

### The Method

In an exemplary method, the delivery sheath 108 is inserted in a patient's vascular system in a conventional manner. The delivery sheath 108 is advanced through a patient's vascular system until the distal end of the delivery sheath 108 reaching the intended target closing site (e.g., a valve or a closing site in a vessel).

The closing device 102 may be releasably mounted adjacent the distal end of the delivery catheter 103 prior to loading the delivery catheter 103 into the delivery sheath 108. For example, the closing device 102 is mounted to the proximal end portion of the delivery catheter 103, and pushed along the delivery catheter 103 to the distal end portion of the delivery catheter 103. The closing device 102 is then loaded into the delivery sheath 108 and advanced though the delivery sheath 108, until the distal end of the delivery catheter 103 is adjacent the valve 4, as seen in FIG. 2.

Alternatively, the delivery catheter 103 is loaded into the delivery sheath 108, without the closing device 102, and advanced though the delivery sheath 108, until the distal end of the delivery catheter 103 is adjacent the valve 4, as seen in FIG. 2. The closing device 102 may be subsequently releasably mounted adjacent the distal end of the delivery catheter 103, in the manner indicated above.

When the distal end of the delivery catheter 103 is adjacent the valve 4, vacuum is applied using the first suction line 106 to draw the valve leaflets 5 into the delivery tool 101, more particularly into the lumen 104 of the delivery catheter 103, as seen in FIG. 3. At any stage of the closing process, contents from the vessel 1 may be drawn out using the second suction line 107.

When the leaflets 5 (i.e., the at least one portion of the valve) are contained in the delivery catheter 103, the release catheter 105 is advanced over the delivery catheter 103, beyond the distal end of the delivery catheter 103, thereby releasing the closing device 102 from the delivery catheter 103. The closing device 102 now partially or completely surrounds the circumference of the leaflets 5.

The leaflets 5 may be in sealed contact against each other, owing to the closing device 102. This may be achieved automatically if the closing device 102 is made or comprises for example of an elastic material, an elastometric material and/or a shape-memory material. Alternatively, the closing device 102 may be closed by the clinician through proximal control of the delivery tool 101. For example, the release catheter 105 may be used to close or tightened the closing device 102 around the leaflets 5.

Once the closing device 102 is in place, the system 100 may be withdrawn.

It will be appreciated that the system 100 may be used for closing a vessel 1 in a patient's vascular system, so that FIG. 2 also illustrates a system 100 for closing a vessel 1 in a patient's vascular system. The system 100 comprises a delivery tool 101 for delivering a closing device 102. The delivery tool 101 comprises a delivery catheter 103 with a lumen 104 configured to receive at least one portion of the vessel 1 in the lumen 104 of the delivery catheter 103; and a closing device 102 configured to be coupled to said at least one portion of the vessel 1 so as to close the vessel 1.

In this embodiment, the at least one portion of the vessel 1 comprises a portion of the wall 3 of the vessel 1. For example, the at least one portion of the vessel 1 comprises a circumferential portion of the vessel 1. The closing device 102 may be configured to be coupled to the wall 3 of the vessel 1 so as to close the vessel 1. Coupling may be effected by bringing the circumference of the vessel wall 3 inwards of the vessel 1 into the delivery tool 101 using the first suction line 106, and closing the vessel 1 with a closing device 102 in the manner described hereinabove in relation to the closure of a valve 4.

Thus, the present disclosure provides improved systems and methods to treat chronic venous disease and chronic venous insufficiency. The present disclosure also provides improved systems and methods to safely and simply close or seal valves and/or vessels in the vascular system of a patient. In particular, the present disclosure provides improved systems and methods to close or seal incompetent vessels, such as varicose or otherwise defective veins.
P Proximal side
D Distal side
1 Vessel
2 Lumen of vessel
3 Wall of vessel
4 Valve
5 Leaflets of valve
100 System
101 Delivery tool
102 Closing device
103 Delivery catheter
104 Lumen of delivery catheter
105 Placement/release catheter
106 First suction line (valve vacuum)
107 Second suction line (vessel vacuum)
108 Delivery sheath

1. A system for closing a valve of a vessel or for closing a vessel in a patient's vascular system, said system comprising:
a delivery tool for delivering a closing device, and comprising a delivery catheter with a lumen configured to receive at least a portion of the valve therein; and
the closing device configured to be coupled to said at least a portion of the valve so as to close the valve.

2. The system of claim 1, wherein the closing device is configured to be releasably mounted to the delivery catheter.

3. The system of claim 1, wherein the closing device is releasably mounted to a distal end of the delivery catheter.

4. The system of claim 1, wherein the closing device is moveable from the proximal end to the distal end of the delivery catheter.

5. The system of claim 1, wherein a circumference of the closing device is adjustable.

6. The system of claim 1, wherein the closing device comprises a sealing ring.

7. The system of claim 1, wherein the closing device comprises an adhesive coating.

8. The system of claim 1, wherein the delivery tool comprises a release device for releasing the closing device from the delivery tool, said release device being configured to slide axially along the delivery catheter.

9. The system of claim 1, wherein the delivery tool comprises a placement device configured to push the closing device from the proximal end of the delivery catheter to the distal end of the delivery catheter, said placement device being configured to slide axially along the delivery catheter.

10. The system of claim 8, wherein the delivery tool comprises an outer catheter concentrically positioned around the delivery catheter.

11. The system of claim 1, wherein the system further comprises a suction tool.

12. The system of claim 11, wherein the suction tool comprises a first suction line arranged and configured to draw at least a portion of the valve into the lumen of the delivery catheter.

13. The system of claim 11, wherein the suction tool comprises a second suction line for drawing contents from the vessel.

14. The system of claim 1, wherein the system comprises a control device for controlling the release device and/or the placement device.

15. A method for closing a valve or a vessel of a patient's vascular system, said method comprising the steps of:
delivering a closing device using a delivery tool comprising a delivery catheter with a lumen configured to receive at least a portion of the valve therein; and
closing the valve using the closing device configured to be coupled to said at least a portion of the valve.

16. The method of claim 15, further comprising the step of releasably mounting the closing device to the delivery catheter.

17. The method of claim 15, further comprising the step of moving the closing device from a proximal end to a distal end of the delivery catheter, using a placement device configured to slide axially along the delivery catheter.

18. The method of claim 15, further comprising the step of releasing the closing device from the delivery tool, using a release device being configured to slide axially along the delivery catheter.

19. The method of claim 17, wherein the delivery tool comprises an outer catheter concentrically positioned around the delivery catheter.

20. The method of claim 15, further comprising the step of drawing at least a portion of the valve into the lumen of the delivery catheter.

21. The method of claim 15, further comprising the step of drawing contents from the vessel.

22. (canceled)

23. (canceled)
